(19) United States
(12) Patent Application Publication
(10) Pub. No.: US 2023/0057186 A1
(43) Pub. Date: Feb. 23, 2023

(54) FERMENTED ONION COMPOSITION

(71) Applicant: Purac Biochem B.V., Gorinchem (NL)

(72) Inventors: David John CHAREST, Lenexa, KS (US); Iancu CATALIN, Gorinchem (NL); Saurabh KUMAR, Lenexa, KS (US); Jarne POSTMUS, Krommerie (NL); Lambertus Henricus , Elisabeth ROOZEN, Gorinchem (NL)

(21) Appl. No.: 17/962,208

(22) Filed: Oct. 7, 2022

Related U.S. Application Data
(63) Continuation of application No. PCT/US2020/027569, filed on Apr. 10, 2020.

(51) Int. Cl. A23L 27/10; A23B 4/12; A23B 4/20; A23L 13/40; A23L 19/00; A23L 2/38; A23L 3/3472; A23L 3/3499; A61K 36/8962

(52) U.S. Cl. CPC A23L 27/105; A23B 4/12; A23B 4/20; A23L 13/428; A23L 19/00; A23L 2/382; A23L 3/3472; A23L 3/3499; A61K 36/8962; A61K 2236/19

(57) ABSTRACT

The invention relates to a fermented onion composition having a dry matter content of at feast 8 wt. %, said composition comprising, per gram of dry matter:
a) 0-150 mg of saccharides selected from fructose, glucose, sucrose and combinations thereof;
b) 5-250 mg of phenols selected from acid equivalent of gallic acid, acid equivalent of ferulic acid, quercetin, kaempferol and combinations thereof;
c) 75-500 mg acid equivalent of organic acid selected from propionic acid, lactic acid, acetic acid and combinations thereof,

The fermented onion composition of the present invention can advantageously be used as an effective, label-friendly food ingredient to increase overall quality of foods.

The invention also provides a method of preparing a fermented onion composition, comprising:
providing an onion substrate containing 10-50 wt. % dry matter and 50-90 wt. % water, wherein at least 40 wt. % of the dry matter is derived from onion;
inoculating the substrate with a micro-organism selected from lactic acid bacteria, Propionibacterium and combinations thereof; and
incubating the inoculated substrate at a temperature in the range of 25-60° C. for at least 12 hours to produce an onion ferment.

### CROSS-REFERENCE TO RELATED PATENT APPLICATIONS

This application is a continuation application filed under 35 U.S.C. § 111(a) claiming the benefit under 35 U.S.C. §§ 120 and 365(c) of International Patent Application No. PCT/US2020/027569, filed on Apr. 10, 2020, the disclosures of all which are incorporated herein by reference in its entirety.

### TECHNICAL HELD OF THE INVENTION

The invention relates to a fermented onion composition having a dry matter content of at least 8 wt. %, said composition comprising, per gram of dry matter:
a) 0-150 mg of saccharides selected from fructose, glucose, sucrose and combinations thereof;
b) 5-250 mg of phenols selected from acid equivalent of gallic acid, acid equivalent of ferulic acid, quercetin, kaempferol and combinations thereof;
c) 75-500 mg acid equivalent of organic acid selected from propionic acid, lactic acid, acetic acid and combinations thereof.

The fermented onion composition of the present invention can advantageously be used as an effective, label-friendly food ingredient to increase overall quality of foods.

The invention further provides a method of preparing a fermented onion composition, comprising:
providing an onion substrate containing 8-50 wt. % dry matter and 50-90 wt. % water, wherein at least 40 wt. % of the dry matter is derived from onion;
inoculating the substrate with a micro-organism selected from lactic acid bacteria, Propionibacterium and combinations thereof; and
incubating the inoculated substrate at a temperature in the range of 25-60° C. for at least 12 hours to produce an onion ferment.

### BACKGROUND OF THE INVENTION

Food preservation involves the action taken to maintain foods with the desired properties or nature for as long as possible. Mechanical, physical, chemical, and microbial effects are the leading causes of food deterioration and spoilage. Based on the mode of action, the major food preservation techniques can be categorized as (1) slowing down or inhibiting chemical deterioration and microbial growth, (2) directly inactivating bacteria, yeasts, moulds, or enzymes, and (3) avoiding recontamination before and after processing

The use of chemicals in foods is a well-known method of food preservation. Wide varieties of chemicals are used in food preservations as additives to control pH, as antimicrobials and antioxidants, and to provide food functionality as well as preservation action. Many legally permitted preservatives in foods are organic acids and esters, including sulfites, nitrites, acetic acid, citric acid, lactic acid, sorbic acid, benzoic acid, sodium diacetate, sodium benzoate, methyl paraben, ethyl paraben, propyl paraben, and sodium propionate. Some of these additives may be perceived as undesirable or less attractive by consumers for instance because they are perceived as “chemical”, “artificial” or “unhealthy”. They may prefer additives that are natural, that can be labelled as a food product instead of an additive, that originate from biological material or that are renewable. Food companies have made good progress in developing products that meet those customer demands, making use of, for instance, vegetables, fruits, herbs and spices or of products fermented with bacteria, yeasts and molds to preserve foods.

Many preservative products are at least partly based on organic acids such as acetic acid, lactic acid and propionic acid. When a weak acid is dissolved in water, equilibrium is established between undissociated acid molecules and charged anions, the proportion of undissociated acid increasing with decreasing pH. The currently accepted theory of preservative action against micro-organisms suggests inhibition via depression of internal cell pH. Undissociated acid molecules are lipophilic and pass readily through the plasma membrane by diffusion. In the cytoplasm, approximately at pH 7.0, acid molecules dissociate into charged anions and protons. These cannot pass across the lipid bilayer and accumulate in cytoplasm, thus lowering pH and inhibiting metabolism. There are several limitations to the value of organic acids as microbial inhibitors in foods:
They are usually ineffective when initial levels of microorganisms are high.
Many microorganisms use organic acids as metabolizable carbon sources.
There is inherent variability in resistance of individual strains.

WO 2018/106109 describes a natural antimicrobial composition comprising a combination of a buffered food acid component and a nitrite source in the form of a cultured vegetable extract.

Santas et al. (Onion. A natural alternative to artificial food preservatives, AgroFood industry hi-tech-September/October (2010) vol 21 n 5) observe that onion is widely used as a food ingredient and it is known as a good source of bioactive compounds, such as sulphur-containing compounds and flavonoids with well-known health beneficial effects, antioxidant and antimicrobial capacities. The authors report that several studies have tested in vitro the effect of organosulfur-containing compounds and have shown that they exhibit marked inhibition against gram positive bacteria of genera Bacillus, Micrococcus, Staphylococcus, Streptococcus, as well as gram negative bacteria, such as Salmonella enteridis or some strains of Escherichia coli. In addition, onion anti-yeast and anti-fungal activity has been mainly attributed to the presence of organosulfur-containing compounds which inhibit the growth of yeast and fungi. The authors further note that the flavonols quercetin and kaempferol are commonly present in notable amounts in onions and are the main non-volatile compounds responsible for their antimicrobial properties. In vitro studies have reported that onion flavonoids can effectively inhibit the growth of gram positive bacteria associated to food spoilage, such as Bacillus cereus, B. subtilis, Staphylococcus aureus, Microcroccus luteus and Listeria monocytogenes.

Dublado et al. (FERMENTED ONION (Allium cepa) JUICE SUPPLEMENTATION TO BROILERS, Journal of Science, Engineering and Technology (2013), 71-77) describes a study in which the effects of fermented onion juice on the growth performance of broilers was investigated. The fermented onion juice was prepared by mixing 1 kg of chopped onion bulbs with 0.5 kg of brown sugar in a pail, covering the pail with a plastic bag with a weight on it. After the 12 hours the weight was removed, and the pail was covered with a manila paper that was tied to the pail with a rubber band. On the seventh day, the juice was collected in a bottle and stored in a cool place.

### SUMMARY OF THE INVENTION

The inventors have discovered that, despite its inherent antimicrobial activity, it is possible to ferment onion juice or onion pulp using Propionibacterium or lactic acid bacteria to further enhance its functional properties as a food ingredient, Onion has a high content of fermentable sugars that can be metabolised into propionic acid (and acetic acid) by Propionibacterium or into lactic acid by lactic acid bacteria. The fermented onion product so obtained has antimicrobial activity due to the presence of antimicrobial phenols that are naturally present in onion as well as the presence of propionate, lactate and/or acetate produced during fermentation. Next to that, the fermented onion product has antioxidant activity and can increase textural, sensorial and other functional properties of food products.

Accordingly the invention provides a fermented onion composition having a dry matter content of at least 8 wt. %, said composition comprising, per gram of dry matter:
a) 0-150 mg of saccharides selected from fructose; glucose, sucrose and combinations thereof;
b) 5-250 mg of phenols selected from acid equivalent of gallic acid, acid equivalent of ferulic acid, quercetin, kaempferol and combinations thereof;
c) 75-500 mg acid equivalent of organic acid selected from propionic acid, lactic acid, acetic acid and combinations thereof.

The saccharide content of the fermented onion composition is substantially lower than the saccharide content of onions due to the fermentative conversion of these saccharides into organic acid. The concentration level of organic acid in the fermented onion composition is much higher than that naturally found in onion or onion juice.

The fermented onion composition of the present invention can advantageously be used as an effective, label-friendly food ingredient to increase overall quality of foods.

The invention also provides a method of preparing a fermented onion composition, comprising:
providing an onion substrate containing 10-50 wt. % dry matter and 50-90 wt. % water, wherein at least 40 wt. % of the dry matter is derived from onion;
inoculating the substrate with a micro-organism selected from lactic acid bacteria, Propionibacterium and combinations thereof; and
incubating the inoculated substrate at a temperature in the range of 25-60° C. for at least 12 hours to produce an onion ferment.

### DETAILED DESCRIPTION OF THE INVENTION

A first aspect of the invention relates to a fermented onion composition having a dry matter content of at least 3 wt. %, said composition comprising, per gram of dry matter:
a) 0-150 mg of saccharides selected from fructose, glucose, sucrose and combinations thereof;
b) 5-250 mg of phenols selected from acid equivalent of gallic acid, acid equivalent of ferulic acid, quercetin, kaempferol and combinations thereof;
c) 75-500 mg acid equivalent of organic acid selected from propionic acid, lactic acid, acetic acid and combinations thereof.

The term “acid” as used herein, unless indicated otherwise, encompasses protonated forms of the acid, dissociated forms of the acid and salts of the acid. Thus, the term “citric acid” encompasses, for instance, C₃H₅O(COOH)₃, C₃H₅O(COOH)₂(COO)⁻, C₃H₅O(COO)³⁻₃, C₃H₅O(COONa)₃ and C₃H₅O(COOH)(COONa)(COO)⁻.

The terminology “mg acid equivalent” as used herein refers to the total amount of a particular acid if the acid were exclusively present in fully protonated from. Thus, 200 mg of sodium propionate equates to 200×74.03/96.06=154.24 mg acid equivalent.

The term “quercetin” as used herein, unless indicated otherwise, encompasses both the aglycone as well as glycosides of quercetin.

The fermented onion composition of the present invention may be provided in the form of a liquid, a paste or a solid (e.g. a powder). Typically, the fermented onion composition has a water content of 0-50 wt. %, more preferably of 0-40 wt. %, more preferably of 0-35 wt. %, most preferably of 0-18 wt. %.

According to a particularly preferred embodiment, the fermented onion composition is a particulate composition, preferably a particulate composition having an average diameter D[4,3] in the range of 100 to 900 μm, more preferably in the range of 200 to 800 μm and most preferably in the range of 300 to 700 μm. Here the ‘average diameter D[4,3]’ refers to the De Brouckere mean diameter or volume mean diameter and can be defined as the weighted average volume, assuming spherical particles of the same volume as the actual particles. This average diameter is calculated by means of the following equation:

D[4,3]=(Σ⁴)/(τ³)

wherein:

$${D}_{i} = mean particle size in size class i ; \\ {n}_{i} = number of particles in size class i$$

In a further preferred embodiment, at least 80 wt. % of the particulate composition has a particle size in the range of 40 to 1500 μm, more preferably in the range of in the range of 100 to 1200 μm, most preferably in the range of in the range of 200 to 1,000 μm. The mass weighted average particle size can suitably be determined using laser diffraction (Malvern Mastersizer 3000).

The combination of components a) to c) (saccharides, phenols and organic acid) typically constitutes at least 40 wt. % of the dry matter that is contained in the composition. More preferably, the combination of components a) to C) constitutes 45-94 wt. % of the dry matter that is contained in the composition. Most preferably, the combination of components a) to C) constitutes 50-89 wt. % of the dry matter that is contained in the composition.

The fermented onion composition typically contains, per gram of dry matter, 0-120 mg, more preferably 0-100 mg and most preferably 0-90 mg of saccharides selected from fructose, glucose, sucrose and combinations thereof.

According to a particularly preferred embodiment, the fermented onion composition additionally comprises a component d) in the form onion acid selected from citric acid, malic acid, tartaric acid, oxalic acid, pyruvic acid, succinic acid and combinations thereof. The concentration levels of the onion acids in the fermented onion composition are comparable to the levels naturally found in onion and onion juice. Per gram of dry matter, the onion acid is preferably present in the fermented onion composition in a concentration of 3-120 mg acid equivalent, more preferably of 5-110 mg acid equivalent, even more preferably of 10-100 mg acid equivalent and most preferably of 15-90 mg acid equivalent.

The combination of components a) to d) (saccharides, phenols, organic acid and onion acid) typically constitutes at least 50 wt. % of the dry matter that is contained in the composition. More preferably, the combination of components a) to d) constitutes 52-95 wt. % of the dry matter that is contained in the composition. Most preferably, the combination of components a) to d) constitutes 55-90 wt. % of the dry matter that is contained in the composition.

In one embodiment of the invention, the composition contains per gram of dry matter 200-800 mg lactic acid equivalent. More preferably, the composition contains per gram of dry matter 300-700 mg lactic acid equivalent. Most preferably, the composition contains per gram of dry matter 400-600 mg lactic acid equivalent.

In another embodiment of the invention the composition contains per gram of dry matter 75-450 mg propionate equivalent. More preferably, the composition contains per gram of dry matter 125-400 ma propionate equivalent. Most preferably, the composition contains per gram of dry matter 175-350 mg propionate equivalent.

Besides propionate, the composition typically contains per gram of dry matter 10-150 mg acetate equivalent, more preferably 20-130 mg acetate equivalent and most preferably 30-110 mg acetate equivalent.

The antimicrobial activity of the fermented onion composition may be further enhanced by using fermentation to not only produce organic acid, but also bacteriocin. The bacteriocin may be one or more bacteriocins selected from the group of nisin, subtilin, cytolysin, variacin, sakacin, pediocin, curvaticin, enterocin and lactacin. More preferably, the bacteriocin may be one or more selected from the group of raisin, sakacin, pediocin and lactacin. Most preferably the bacteriocin is nisin. According to a preferred embodiment, the composition contains per gram of dry matter 5,000-500,000 IU nisin equivalent activity. More preferably, the composition contains per gram of dry matter 10,000-400,000 IU nisin equivalent activity. Most preferably, the composition contains per gram of dry matter 50,000-300,000 IU nisin equivalent activity.

Since lactic acid and bacteriocin can both be produced by lactic acid bacteria, in a preferred embodiment, the fermented onion composition contains both lactic acid and bacteriocin. In an even more preferred embodiment, the fermented onion composition contains lactic acid and nisin.

The fermented onion composition typically has a total nitrogen content of 0.5-35 mg per gram of dry matter, more preferably a total protein content of 2-30 mg per gram of dry matter and most preferably a total protein content of 3-25 mg per gram of dry matter. The total protein content is suitably determined using the Kjeldahl method.

Preferably, at least 90 wt. %, more preferably at least 95 wt. % of the protein contained in the fermented onion composition is onion protein.

According to a further preferred embodiment at least 60 wt. %, more preferably at least 70 wt. % and most preferably at least 80 wt. % of the dry matter contained in the fermented onion composition is derived from onion.

The fermented onion composition of the present invention typically contains appreciable amounts of components that are naturally present in onions, such as onion acids, phenols (including flavonols), sulphur-containing compounds, aldehydes, ketones.

The fermented onion composition preferably comprises per gram of dry matter, 0.05-30 mg acid equivalent of citric acid, more preferably 0.15-25 mg acid equivalent of citric acid and most preferably 0.25-20 mg acid equivalent of citric acid.

The fermented onion composition preferably comprises per gram of dry matter, 0.2-30 mg acid equivalent of malic acid, more preferably 0.5-25 mg acid equivalent of malic acid and most preferably 1-20 mg acid equivalent of malic acid.

The fermented onion composition preferably comprises per gram of dry matter, 1-150 mg acid equivalent of gallic acid, more preferably 3-125 mg acid equivalent of gallic acid and most preferably 5-100 mg acid equivalent of gallic acid.

The fermented onion composition preferably comprises per gram of dry matter 0.05-8 mg acid equivalent of ferulic acid, more preferably 0.15-7 mg acid equivalent of ferulic acid and most preferably 0.25-6 mg acid equivalent of ferulic acid.

Preferably, the fermented onion composition contains per gram of dry matter 0.2-30 mg quercetin, More preferably, the fermented onion composition contains per gram of dry matter 0.5-25 mg quercetin. Most preferably, the fermented onion composition contains per gram of dry matter 1-20 mg quercetin. Here the amount of quercetin refers to the aglycone form of quercetin.

Preferably, the fermented onion composition contains per gram of dry matter 0.05-8 mg kaempferol. More preferably, the fermented onion composition contains per gram of dry matter 0.15-7 mg kaempferol. Most preferably, the fermented onion composition contains per gram of dry matter 0.25-6 mg kaempferol.

Cellular material from the micro-organisms used in the preparation of the fermented onion composition is typically present in said composition. Accordingly, in a preferred embodiment, the composition contains cellular material from lactic acid bacteria and/or Propionibacterium.

According to another preferred embodiment, the bulk of dry matter contained the fermented onion composition is water-soluble. Preferably, at least 80 wt. %, more preferably at least 90 wt. % and most preferably at least 95 wt. % of the composition is dissolved at a temperature of 20° C. when the composition is diluted with distilled water to a dry matter content of 5 wt. %.

The fermented onion composition of the present invention is preferably obtainable by, even more preferably obtained by the preparation method described below.

Another aspect of the invention relates to a method of preparing a fermented onion composition, comprising:
providing an onion substrate containing 10-50 wt. % dry matter and 50-00 wt. % water, wherein at least 40 wt. % of the dry matter is derived from onion;
inoculating the substrate with a micro-organism selected from lactic acid bacteria, Propionibacterium and combinations thereof; and
incubating the inoculated substrate at a temperature in the range of 25-60° C. for at least 12 hours to produce an onion ferment.

According to a particularly preferred embodiment, the aforementioned preparation method yields a fermented onion composition as defined herein before.

The onion substrate employed in the preparation method preferably comprises onion juice, onion pulp or a combination thereof. Onion juice may be provided in the form of concentrated onion juice of onion juice powder. Onion pulp may suitably be provided in the form of onion powder. The powder or concentrate may be diluted with water to obtain onion substrate with 10-50% dry matter as a starting material for the fermentation.

In a preferred embodiment, at least 50 wt. %, more preferably at least 70 wt. % and most preferably at least 80 wt. % of the dry matter of the onion substrate is derived from onion.

Preferably, at least 80 wt. % of the dry matter of the onion substrate is provided by onion juice and/or onion pulp. More preferably, at least 80 wt. % of the dry matter of the onion substrate is provided by onion juice, most preferably by concentrated onion juice having a water content of less than 40 wt. %.

The onion substrate typically contains, calculated by weight of dry matter, at least 40 wt. %, more preferably at least 50 wt. % and most preferably at least 55 wt. % of saccharides selected from fructose, glucose, sucrose and combinations thereof.

In an advantageous embodiment of the present invention the onion substrate has been treated with invertase prior to inoculation. Invertase treatment converts sucrose into glucose and fructose which may be more effectively metabolised by certain strains of lactic acid bacteria and/or Propionibacterium.

The total nitrogen content of the onion substrate, calculated by weight of dry matter, preferably lies in the range of 0.15-3.5 wt. %, more preferably in the range of 0.2-3.0 wt. % and most preferably in the range of 0.3-2.5 wt. %. The total protein content of the onion substrate is suitably determined using the Kjeldahl method.

Onion acid selected from citric acid, malic acid, gallic acid and combinations thereof is preferably present in the onion substrate in a concentration, per gram of dry matter, of 3-120 g acid equivalent, more preferably 5-110 mg acid equivalent, even more preferably of 10-100 mg acid equivalent and most preferably of 15-90 mg acid equivalent.

Preferably, the onion substrate contains, calculated by weight of dry matter, 0.02-3 wt. % quercetin, more preferably 0.05-2.5 wt. % quercetin and most preferably 0.1-2 wt. % quercetin. Here the amount of quercetin refers to the aglycone form of quercetin.

The onion substrate preferably contains 11-45 wt. % dry matter and 89-55 wt. % water. More preferably, the onion substrate contains 12-40 wt. % dry matter and 88-60 wt. % water.

The onion substrate preferably has been pasteurised or sterilised before the inoculation. Most preferably, the onion substrate is sterile when it is inoculated with the lactic acid bacteria and/or Propionibacterium.

Typically, the onion substrate is inoculated with at least 0.1% w/w of a pre-culture of the micro-organism. More preferably the onion substrate is inoculated with 0.5-40% w/w of a pre-culture, most preferably 1.0-30% w/w of a pre-culture of the micro-organism.

The inoculated substrate is preferably incubated at a temperature in the range of 20-70° C., more preferably at a temperature of 25-65° C. for at least 12 hours to produce the onion ferment.

According to another preferred embodiment, the inoculated substrate is incubated under anaerobic conditions.

Typically, incubation is continued until the incubated substrate contains, calculated by dry matter, 10-50 wt. % acid equivalent of organic acid selected from propionic acid, lactic acid and combinations thereof.

In one embodiment of the present invention base is added during and/or after incubation so as to neutralise at least a part of the organic acid that is formed during incubation. Preferably, sufficient base is added to increase pH of the onion ferment to 5.0 or more, more preferably to increase pH of the onion ferment to 5.5-7.5

After incubation, the onion ferment is preferably sterilised, e.g. by means of UHT sterilisation.

The onion ferment is preferably concentrated to a dry matter content of at least 50 wt. %, more preferably of at least 60 wt. % after incubation.

According to a particularly preferred embodiment, the onion ferment is dried to produce a dry powder having a water content of not more than 18 wt. %, preferably of not more than 15 wt. %. Drying techniques that may suitably be employed are spray drying, drum drying and freeze drying. After drying, the dried onion ferment may suitably be subjected to additional processing steps such as milling, sieving and granulation. Most preferably, the onion ferment is dried by means of spray drying

A further aspect of the invention relates to a process of preparing a food product or a beverage, said process comprising combining the fermented onion composition according to the present invention with one or more other ingredients. The fermented onion composition of the present invention is particularly suitable for preserving food products such as meat products, savoury products such as soups and sauces, salads and (vegetable) beverages.

Preferably, the process of preparing a food product or a beverage comprises incorporating the fermented onion composition in a concentration of 0.1-10%, more preferably in a concentration of 0.2-5% by weight of the final food product or the final beverage.

Yet another aspect of the invention relates to the use of the fermented onion composition of the present invention as a food preservative. This use typically comprises mixing of the fermented onion composition with one or more other ingredients of the product that requires preservation.

Yet another aspect of the invention relates to the use of the fermented onion composition to prevent microbial spoilage of food products. Yet another aspect of the invention relates to the use of the fermented onion composition to improve the sensory properties of meat products.

The invention is further illustrated by the following non-limiting examples.

### EXAMPLES

### Example 1

A fermented onion composition according to the invention was produced by treating onion juice with invertase to convert sucrose into glucose and fructose, followed by fermentation using B. coagulans (deposited by Corbion NV at the DSMZ depository institution (German collection of microorganisms and cell cultures) 20 Mar. 2020 and having a deposit number DSM 33469) to convert the fermentable sugar's in the treated onion juice into lactate. The results shown below were obtained from 6 repetitions of this experiment.

A specification of the onion juice concentrate is shown in Table 1

TABLE 1

| Dry matter | 67-76 | wt. % |
|---|---|---|
| Brix (20° C.) | 72-74° | |
| pH | 4.2-5.1 | |
| Solubility | Fully soluble in water | |
| Glucose | 70-150 | g/kg |
| Fructose | 60-190 | g/kg |
| Sucrose | 1-28 | g/kg |
| Protein | 58-108 | g/kg |

To prepare an onion juice stock solution, 3300 grams of onion juice concentrate were diluted with 6700 grams of demineralised water. Next, the diluted onion juice was UHT sterilised. After the sterilised onion juice dilution had cooled down to ambient temperature, it was used to prepare onion juice medium. For this, 2500 g of onion juice stock solution was mixed with 2245 g of demineralised water. The pH of the resulting medium was ˜4.6. Next, 5 ml of an invertase solution was added and incubated for 30 minutes at 54° C. after which all sucrose had been converted to glucose and fructose.

The sugar composition of a typical onion juice medium after invertase treatment is shown in Table 2.

TABLE 2

| | g/kg |
|---|---|
| Glucose | 21-24 |
| Fructose | 33-36 |

Next, the pH of the invertase treated onion juice medium was adjusted to 6.4 by adding a 40% caustic solution of Ca(OH)₂ and NaOH (50/50 w/w ratio), followed by inoculation with 250 ml of an inoculum containing B. coagulans (OD₅₀₀ 6-7).

Fermentation was conducted under anaerobic conditions in a stirred fermenter at a temperature of 54° C. During fermentation the temperature of the fermentation broth was maintained within the range of 50-56° C. The aforementioned caustic solution was used to maintain pH within the range of 6-6.8.

After 30 hours of fermentation, a sample was taken from the fermentation broth and analysed. The results of the analysis are shown in Table 3.

TABLE 3

| | g/kg |
|---|---|
| Lactic acid | 51.2-56.4 |
| Acetic acid | 0.0-0.93 |
| Propionic acid | 0.0 |
| Formic acid | 0.19-0.61 |
| Succinic acid | 0.16-0.24 |
| Glucose | 0.5-0.8 |
| Fructose | 0.5-0.9 |
| Sucrose | 0.0 |
| Total nitrogen | 1.8-2.2 |

### Example 2

A fermented onion composition according to the invention was produced by treating onion juice with invertase to convert sucrose into glucose and fructose, followed by fermentation using P. acidipropionici (deposited by Corbion NV at the DSMZ depository institution (German collection of microorganisms and cell cultures) 20 March 2020 and having a deposit number DSM 33468) to convert the fermentable sugars in the treated onion juice into propionate and acetate. The results shown below were obtained from 7 repetitions of this experiment.

3300 grams of the same onion juice as in Example 1 were diluted with 6700 grams of demineralised 10 water. Next, the diluted onion juice was UHT sterilised.

After the sterilised onion juice dilution had cooled down to ambient temperature an onion juice fermentation medium was prepared and an invertase solution was added. The composition of the fermentation medium is shown in Table 4.

TABLE 4

| | | |
|---|---|---|
| Onion juice dilution | 2500 | g |
| Yeast extract paste (50% w/w) | 250 | g |
| Trace solution ¹ | 0.625 | mL |
| Vitamin solution ² | 50 | mL |
| Panthotenate solution (500 mg/L) | 50 | mL |
| Demineralised water | 1900 | g |
| Invertase solution | 5 | mL |

¹ FeCl₃*6H₂O (190 g/L), MnCl₂ (120 g/L), CoCl₂ (100 g/L) and ZnCl₂ (30 g/L)
² Biotin, thiamine, PABA (each 100 mg/L)

The onion juice medium had pH of about 4.6. After 2 hours of incubation at 35° C. all sucrose had been converted to glucose and fructose.

Next, the pH of the invertase treated onion juice dilution was adjusted to 7 by adding a 40% caustic solution of Ca(OH)₂ and NaOH (50/50 w/w ratio), followed by inoculation with 250 ml of an inoculum containing P. acidipropionici (OD₅₀₀ 30-40).

Fermentation was conducted under anaerobic conditions in a stirred fermenter. During fermentation temperature of the fermentation broth was maintained at 30-38° C. and caustic solution (20% Ca(OH)₂) was added to maintain pH within the range of 6-8.

After 40 hours of fermentation, a sample was taken from the fermentation broth and analysed. The results of the analysis of a typical fermentation on onion juice medium are shown in Table 5.

TABLE 5

| | g/kg |
|---|---|
| Lactic acid | 0.05-0.15 |
| Acetic acid | 5.3-9.5 |
| Propionic acid | 21.9-27.3 |
| Formic acid | 0.2-1.0 |
| Glucose | 0.0 |
| Fructose | 0.0 |
| Sucrose | 0.0 |
| Total nitrogen | 3.1-3.9 |

### Example 3

The effect of the use of fermented onion composition on purge loss, cooked meat pH and color in a turkey meat formulation was investigated. Laboratory scale tests were performed using 13 mm pieces of turkey breast to which 20% w/w of a brine solution was added.

Five different brine solutions were used containing salt, cane sugar, carrageenan. Four of the tested brine solutions additionally contained the fermented onion composition of Example 1 or Verdad powder N20 (ex Corbion, the Netherlands). The concentrations of salt, cane sugar and carrageenan calculated by weight of the final meat product were 1.6%, 0.5% and 0.4%, respectively. The concentrations of the fermented onion composition and the Verdad powder, also calculated by weight of the final meat product, are shown in Table 6; the final concentrations of lactate were similar for Onion 1 and N20-1 (lower dosage) and Onion 2 and N20-2 (higher dosage).

TABLE 6

| | Control | Onion 1 | Onion 2 | N20-1 | N20-2 |
|---|---|---|---|---|---|
| Fermented onion | | 1.18% | 1.62% | | |
| Verdad powder N20 | | | | 0.83% | 1.14% |

The brine solutions were prepared by adding the brine ingredients to water, followed by five minutes of mixing. Next, the brine solutions were added to the meat pieces, and vacuum tumbled for two hours. After tumbling, the meat batters were vacuum stuffed into 60 mm plastic, high barrier casings and thermally processed to an internal temperature of 72° C.; following the cooking schedule outlined in Table 7. The meat products so obtained were chilled overnight to an internal temperature of 2° C.

TABLE 7

| | | Chamber Temp. | Relative Humidity |
|---|---|---|---|
| Time, Minutes | Stage | (in ° C.) | (in %) |
| 5 | Cold water shower | — | — |
| 30 | Cook | 60.0 | ≥98 |
| 30 | Cook | 65.6 | ≥98 |
| 30 | Cook | 71.1 | ≥98 |
| 30 | Cook | 76.7 | ≥98 |
| time until internal meat | Cook | 90.6 | ≥98 |
| temperature 72.8° C. | | | |
| 30 | Cold water shower | — | — |

The pH of the raw meat, brines, meat batters and cooked meat products was determined. Also the purge loss and color were determined for each of the meat products. The purge loss equals the weight difference between the turkey meat product before and after cooking (without unbound liquid), divided by the weight of the meat product before cooking. Color was measured according to the CIELAB standard. The results are provided in Table 8 and show that the fermented onion performs better than Verdad powder N20.

TABLE 8

| | Control | Onion 1 | Onion 2 | N20-1 | N20-2 |
|---|---|---|---|---|---|
| Raw Meat pH | 5.81 | 5.81 | 5.81 | 5.81 | 5.81 |
| Brine pH | 7.64 | 6.54 | 6.19 | 6.55 | 6.51 |
| Meat Batter pH | 5.87 | 5.98 | 5.99 | 5.90 | 5.89 |
| Cooked Meat pH | 6.11 | 5.73 | 5.65 | 5.76 | 5.64 |
| Purge toss (% w/w) | 11.8 | 9.9 | 11.1 | 11.0 | 11.4 |
| Color | | | | | |
| L | 84.0 | 80.3 | 78.9 | 82.9 | 82.4 |
| a | 3.00 | 5.35 | 5.79 | 3.49 | 3.88 |
| b | 13.2 | 16.7 | 18.2 | 13.1 | 13.1 |

| | |
|---|---|
| Remarks | meat is slightly darker and |
| | slightly more red(/yellow) |
| | than control and N20-1 and |
| | N20-2 |

### Example 4

The antimicrobial efficacy of fermented onion compositions was investigated in cooked chicken rolls which were inoculated with a cocktail of lactic acid bacteria (LAB), relevant to meat spoilage.

The growth of the lactic acid bacteria was followed during storage, in meat samples to which onion ferments had been added (test product), compared to samples to which a commercial preservative product had been added (reference product) and to a sample without added preservative (control).

### Meat Samples Preparation

Chicken roll samples (Table 9) were prepared using 30 mm chicken fillet and 18% brine injection. Meat samples were tumbled for 1 hour after all ingredients had been mixed in. Linear cooking was conducted to reach 72° C. in the core of the rolls.

TABLE 9

| | | | Concentration of actives | | |
|---|---|---|---|---|---|
| No | Sample | Dose levels | Lactate | Acetate | Propionate |
| 1 | Control | — | — | — | — |
| 2 | Verdad Opti.Powder N70* | 1.88% | 0.77% | 0.48% | 0.18% |
| 3 | Fermented onion composition | 1.42% | 0.77% | 0.48% | 0.17% |
| | from Example 1 + | 0.9% | | | |
| | Fermented onion composition | | | | |
| | from Example 2 + | | | | |
| | Neutralized vinegar powder** | | | | |
| | | [symbol] | | | |

*ex Corbion (Gorinchem, the Netherlands)
**prepared by adding 50% NaOH solution to concentrated vinegar up to neutral pH and spray drying the product [symbol]
indicates data missing or illegible when filed

Cooked meat samples were inoculated with a 6 strains LAB cocktail in order to reach a bacterial concentration of 3 Log CFU/g. The LAB cocktail contained lactic acid bacteria obtained from spoilt meat and are known to be relatively resistant to organic acids. The cooked meat samples were kept at 7° C. The LAB growth was followed in time by retrieving and plating the bacterial cells from approx. 20 g sample. Duplicate samples were used at each time point.

### Results

The pH and water activity (A) of the different samples are depicted in Table 10.

TABLE 10

| No | Sample | pH | A_{w} |
|---|---|---|---|
| 1 | Control | 6.19 | 0.98 |
| 2 | Reference: Verdad Opti.Powder N70 | 5.76 | 0.975 |
| 3 | Test: Fermented onion composition from Example 1 + Same from | 5.93 | 0.974 |
| | Example 2 + Neutralized vinegar powder | | |

The water activity of the samples with the reference product and test product are very similar, whereas the water activity of the control is slightly higher. Both addition of the reference product and the test product led to a decrease of the pH, although the sample with the test product was almost 0.2 units higher. This may be due to a higher buffer capacity of the test product.

The results from the bacterial growth measurements are shows in Table 11.

TABLE 11
(log CFU/gram)

| | weeks | | | | | | | | | |
|---|---|---|---|---|---|---|---|---|---|---|
| No | 0 | 2 | 5 | 7 | 9 | 12 | 14 | 16 | 20 | 26 |
| 1 | 1.8 | 2.4 | 5.5 | 7.4 | #N/A | #N/A | 9.0 | 9.2 | 9.1 | 9.2 |
| 2 | 1.8 | 2.0 | 4.2 | 5.0 | 6.7 | #N/A | 8.0 | 8.5 | 8.9 | 9.0 |
| 3 | 1.8 | 2.1 | 3.4 | 4.7 | 5.7 | 7.0 | 7.3 | 8.0 | 8.6 | 9.0 |

Both Verdad Opt. Powder N70 and the test product containing onion ferments inhibited the growth of the resistant LAB in the chicken rolls as compared to the Control. The test product performed slightly better, in spite of the higher pH value of the chicken rolls.

1. A fermented onion composition having a dry matter content of at least 8 wt. % and comprising, per gram of dry matter:
(a) 0-150 mg of saccharides selected from fructose, glucose, sucrose and combinations thereof; and
(b) 75-800 mg acid equivalent of organic acid selected from propionic acid, lactic acid, acetic acid and combinations thereof.

2. The fermented onion composition according to claim 1, having a water content of 0-50 wt. %.

3. The fermented onion composition according to claim 1, further comprising (c) 3-120 mg acid equivalent of onion acid selected from citric acid, malic acid, tartaric acid, oxalic acid, pyruvic acid, succinic acid and combinations thereof.

4. The fermented onion composition according to claim 1, wherein the composition comprises 200-800 mg lactic acid equivalent per gram of dry matter.

5. The fermented onion composition according to claim 1, wherein the composition comprises 75-450 mg propionate equivalent per gram of dry matter.

6. The fermented onion composition according to claim 1, having a total nitrogen content of 1.5-35 mg per gram of dry matter.

7. The fermented onion composition according to claim 1, comprising, per gram of dry matter:
(a) 0.05-30 mg acid equivalent of citric acid;
(b) 0.2-30 mg acid equivalent of malic acid; and
(c) 1-150 mg acid equivalent of gallic acid.

8. The fermented onion composition according to claim 1, comprising cellular material from lactic acid bacteria and/or Propionibacterium.

9. A method of preparing a fermented onion composition, comprising:
(a) obtaining an onion substrate comprising 10-50 wt. % dry matter and 50-90 wt. % water, wherein at least 40 wt. % of the dry matter is derived from onion;
(b) inoculating the substrate with a micro-organism selected from lactic acid bacteria, Propionibacterium and combinations thereof; and
(c) incubating the inoculated substrate at a temperature of 25-60° C. for at least 12 hours to produce an onion ferment.

10. The method according to claim 9, wherein the dry matter of the onion substrate is provided by onion juice and/or onion pulp.

11. The method according to claim 9, for producing a fermented onion composition according to claim 1.

12. A process of preparing a food product or a beverage, the process comprising combining the fermented onion composition according to claim 1 with one or more other ingredients.

13. A method of preserving food, comprising adding the fermented onion composition according to claim 1 to the food.
